(19) United States
(12) Patent Application Publication
(10) Pub. No.: US 2007/0299137 A1
(43) Pub. Date: Dec. 27, 2007

(54) N-Acylated Derivatives Of Dicarboxylic Acids With Amino Acids And Vegetable Protein Hydrolsates And Their Use In Cosmetics And Pharmaceuticals

(76) Inventors: Palmiro Comini, Castiglione Delle Stiviere (IT); Marina Lenzini, Pero (IT)

Correspondence Address: YOUNG & THOMPSON, 745 SOUTH 23RD STREET, 2ND FLOOR, ARLINGTON, VA, 22202, US

(21) Appl. No.: 11/658,732

(22) PCT Filed: Jul. 26, 2005
(86) PCT No.: PCT/EP05/08109

§ 371 Date: Apr. 20, 2007

(30) Foreign Application Priority Data
Jul. 30, 2004 (IT) MI2004A001567

(51) Int. Cl. A61K 31/19; A61P 17/00; C07C 69/34; C07D 207/12

(52) U.S. Cl. 514/574; 548/531; 560/190

(57) ABSTRACT

Compounds of formula (I) 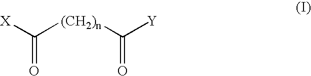 in which: n is an integer from 2 to 16;
X and Y, which can be the same or different, are OM or a residue of formula MOOCHRNH— wherein R is the residue of a natural alpha-amino acid and M is hydrogen or a non toxic cation, or X and/or Y are a —NH—P residue, wherein P is a polypeptide deriving from the hydrolysis of vegetable proteins, with the proviso that at least one of X and Y is different from OH and that X and Y are not both —NHCH₂—COOK groups when n is 7. The compounds of the invention are useful as active ingredients in cosmetic, pharmaceutical and dermopharmaceutical formulations.

The present invention relates to N-acylated derivatives of aliphatic dicarboxylic acids with amino acids or protein hydrolysates and their use in cosmetic, dermopharmaceutical and pharmaceutical formulations.

### TECHNOLOGICAL BACKGROUND

Azelaic acid is used at present in the cosmetic and dermopharmaceutical fields, both in the free and the salified or esterified form.

Azelaic acid is a dicarboxylic fatty acid characterized by crystalline structure and high melting point, both in the free and salified form, which involves problems during the formulation of cosmetic or pharmaceutical products, due to its poor processability.

Azelaic acid is a 5-alpha-reductase inhibitor used in cosmesis as a sebum normalizing agent, in the treatment of acne and as a skin peeling. Some of said applications require high concentrations of azelaic acid in the formulation, up to approximately 20%.

Monocarboxylic fatty acids acylated with amino acids or vegetable hydrolysates are known and used in cosmesis, as they quickly cross the skin first cell layer thanks to their amphiphilic structure, thereby acting as carriers for various active principles.

### DISCLOSURE OF THE INVENTION

It has now been found that derivatives of dicarboxylic acids with amino acids and/or peptides from vegetable protein hydrolysates have improved chemical-physical characteristics which make the preparation of cosmetic and pharmaceutical formulations easier.

The compounds of the invention have the following formula (I)

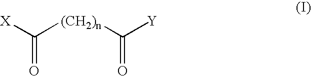

in which:

n is an integer from 2 to 16;

X and Y, which can be the same or different, are OM or a residue of formula MOOCHRNH— wherein R is the residue of a natural alpha-amino acid and M is hydrogen or a non toxic cation, or X and/or Y are a —NH—P residue, wherein P is a polypeptide deriving from the hydrolysis of vegetable proteins, with the proviso that at least one of X and Y is different from OH and that X and Y are not both —NHCH₂—COOK groups when n is 7.

n is preferably an integer from 2 to 16, most preferably 7 (azelaic acid derivatives). Examples of other preferred dicarboxylic acids include octadecendicarboxylic acid, suberic acid, pimelic acid and sebacic acid.

R is preferably hydrogen or the residue of the amino acids arginine (3-(aminoiminomethyl)-propyl), glutamic acid (2-carboxy-propyl) optionally salified, hydroxyproline (in which case the R and NH groups form together a 4-hydroxy-2-pyrrolidino ring), proline (in which case the R and NH groups form together a 2-pyrrolidino ring).

Examples of vegetable protein hydrolysates P comprise soy, oat, wheat and sweet almond protein hydrolysates and other protein hydrolysates of cosmetically interesting plants. Said hydrolysates are commercially available or can be prepared according to known procedures.

Examples of compounds according to the invention include compounds of formula (I) in which:
both X and Y are a residue of formula MOOCHRNH— as defined above;
both X and Y are a residue of formula NH—P as defined above;
one of X and Y is a residue of formula NH—P and the other is a residue of formula MOOCHRNH—, as defined above;
one of X or Y is a residue of forrnula MOOCHRNH— and the other is OM;
one of X or Y is a residue of formula NH—P and the other is OM.

The compounds of the invention can be prepared by acylation of dicarboxylic acids according to conventional methods, for example by activation of one or both carboxylic groups in the form of acid chloride or mixed anhydride and subsequent reaction with the suitable stoichiometric amounts of the amino acids, which can optionally be protected when they contain functional groups which could interfere with the desired reaction. The reaction or reactions, in case X and Y are different from each other, can be carried out in suitable sequence in water and the reaction product can then be salified and purified, or the reaction mixture, consisting of an aqueous solution of the compound, can be used directly, optionally adjusting pH by addition of acids or buffering agents.

The compounds of the invention are completely or partially soluble even at acid pH and are in the amorphous pseudo-plastic form, have low melting point, are highly water-soluble and therefore easy to formulate into cosmetic and pharmaceutical products. Furthermore, they are toxicologically safe and have improved skin tolerability compared with azelaic acid.

The dicarboxylic acids acylated derivatives of formula I, in particular those of azelaic acid, have marked bioavailability and are particularly useful as active ingredients in the sebum normalizing treatments, in the treatment of acne and dandruff, for skin whitening, in the treatment of dry skin, red skin, and alopecia and disorders of the scalp and hair.

For the envisaged uses, the compounds of the invention will be formulated according to conventional techniques and excipients in suitable pharmaceutical and cosmetic forms such as creams, lotions, gel, foams and similar topical forms.

The following examples illustrate the invention in greater detail.

### EXAMPLE 1

Potassium azeloyl diglycinate is prepared by reacting 150 g of azeloyl dichloride with 100 g of glycine in 355 g of distilled water, keeping pH at 9-11 with 187 g of 40% potassium hydroxide.

Azeloyl dichloride and 40% potassium hydroxide are added in small portions, to prevent temperature from exceeding 75° C. The mixture is reacted at pH 9-9.5 for 2 hours keeping the temperature at 65-75° C.

After completion of the reaction, pH is adjusted to 7-7.5 with lactic acid.

The product has the following characteristics:

| | |
|---|---|
| Appearance at 20° C.: | Clear liquid |
| Color: | Colourless to pale yellow |
| Active substance: | Approx. 30% |

### EXAMPLE 2

Sodium azeloyl glutamate is prepared by reacting 100 g of azeloyl dichloride with 167 g of monosodium glutamate hydrate dissolved in 341 g of distilled water. Azeloyl dichloride is added in small portions, to prevent temperature from exceeding 75° C. During the reaction, pH is kept at 9-11 by addition of small portions of 30% sodium hydroxide for a final total of 120 g. After completion of the addition, the mixture is reacted for two hours at approx. 70° C, then pH is adjusted to 7-7.5 with lactic acid. The resulting product has the following characteristics:

| | |
|---|---|
| Appearance at 20° C.: | Clear liquid |
| Color: | Colourless to pale yellow |
| Active substance: | Approx. 30% |

### EXAMPLE 3

Potassium azeloyl glutamyl glicine is prepared by reacting 100 g of Azeloyl dichloride with 33.5 g of glycine and 83.5 g of monosodium glutamate hydrate in 338 g of distilled water. Azeloyl dichloride is added in small portions, to prevent temperature from exceeding 75° C. During the reaction, pH is kept at 9-11 by addition of small portions of 40% potassium hydroxide for a final total of 120 g. After completion of the addition, the mixture is reacted for two hours at approx. 70° C., then pH is adjusted to 7-7.5 with lactic acid. The resulting product has the following characteristics:

| | |
|---|---|
| Appearance at 20° C.: | Clear liquid |
| Color: | Colourless to pale yellow |
| Active substance: | Approx. 30% |

### EXAMPLE 4

Potassium azeloyl proline arginine is obtained by reacting 100 g of Azeloyl dichloride with 51 g of proline and 77 g arginine base in 298 g of distilled water. Azeloyl dichloride is added in small portions, to prevent temperature from exceeding 75° C. During the reaction, pH is kept at 9-11 by addition of small portions of 40% potassium hydroxide for a final total of 187 g. After completion of the addition, the mixture is reacted for two hours at approx. 70° C., then pH is adjusted to 7-7.5 with lactic acid. The resulting product has the following characteristics:

| | |
|---|---|
| Appearance at 20° C.: | Clear liquid |
| Color: | Colourless to pale yellow |
| Active substance: | Approx. 30% |

### EXAMPLE 5

Potassium azeloyl proline hydrolyzed wheat protein is obtained by reacting 100 g of Azeloyl dichloride with 58.5 g of hydroxyproline and 642 g of wheat hydrolysate with 25% active substance. Azeloyl dichloride is added in small portions, to prevent temperature from exceeding 75° C. During the reaction, pH is kept at 9-11 by addition of small portions of 40% potassium hydroxide for a final total of 187 g. After completion of the addition, the mixture is reacted for two hours at approx. 70° C., then pH is adjusted to 7-7.5 with lactic acid. The resulting product has the following characteristics:

| | |
|---|---|
| Appearance at 20° C.: | Clear liquid |
| Color: | Colourless to pale yellow |
| Active substance: | Approx. 30% |

### EXAMPLE 6

Azeloyl bis hydrolyzed soy protein is obtained by reacting 100 g of Azeloyl dichloride with 1.285 g of a soy hydrolysate with 25% of active substance. Azeloyl dichloride is added in small portions, to prevent temperature from exceeding 75° C. During the reaction, pH is kept at 9-11 by addition of small portions of 30% sodium hydroxide for a final total of 120 g. After completion of the addition, the mixture is reacted for two hours at approx. 70° C., then pH is adjusted to 7-7.5 with lactic acid. The resulting product has the following characteristics:

| | |
|---|---|
| Appearance at 20° C.: | Clear liquid |
| Color: | Colourless to pale yellow |
| Active substance: | Approx. 30% |

### EXAMPLE 7

Potassium Azeloyl mono glycine is obtained by reacting 100 g of azeloyl mono chloride with 34 g of glycine dissolved in 76 g of distilled water. Azeloyl dichloride is added in small portions, to prevent temperature from exceeding 75° C. During the reaction, pH is kept at 9-11 by addition of small portions of 40% potassium hydroxide for a final total of 120 g. After completion of the addition, the mixture is reacted for two hours at approx. 70° C., then pH is adjusted to 7-7.5 with lactic acid. The resulting product has the following characteristics:

| | |
|---|---|
| Appearance at 20° C.: | Clear liquid |
| Color: | Colourless to pale yellow |
| Active substance: | Approx. 30% |

### EXAMPLE 8

Sodium Azeloyl bis hydrolysed wheat amino acid is obtained by reacting 100 g of Azeloyl dichloride with 427 g of mixture of soy amino acids with 25% of active substance in 682 g of distilled water. Azeloyl dichloride is added in small portions, to prevent temperature from exceeding 75° C. During the reaction, pH is kept at 9-11 by addition of small portions of sodium hydroxide al 30% for a final total of 180 g. After completion of the addition, the mixture is reacted for two hours at approx. 70° C., then pH is adjusted to 7-7.5 with lactic acid. The resulting product has the following characteristics:

| | |
|---|---|
| Appearance at 20° C.: | Clear liquid |
| Color: | Colourless to pale yellow |
| Active substance: | Approx. 30% |

### EXAMPLE 9

Sodium Azeloyl mono hydrolysate oat amino acid is obtained by reacting 100 g of Azeloyl dichloride with 213 g of mixture of soy amino acids with 25% of active substance in 33 g of distilled water. Azeloyl dichloride is added in small portions, to prevent temperature from exceeding 75° C. During the reaction, pH is kept at 9-11 by addition of small portions of sodium hydroxide al 30% for a final total of 120 g. After completion of the addition, the mixture is reacted for two hours at approx. 70° C., then pH is adjusted to 7-7.5 with lactic acid. The resulting product has the following characteristics:

| | |
|---|---|
| Appearance at 20° C.: | Clear liquid |
| Color: | Colourless to pale yellow |
| Active substance: | Approx. 30% |

Some formulation examples according to the invention are reported in the following.

### Formulation Example 1

Whitening cosmetic cream

| | | |
|---|---|---|
| 1 | Aqua | to 100% |
| 2 | Octyl Palmitate | 15% |
| 3 | Cetearyl Alcohol | 3.5% |
| 4 | Gliceryl Stearate | 3.5% |
| 5 | Potassium Palmitoyl Hydrolyzed Wheat Protein | 2.5% |
| 6 | Jojoba Oil | 3.0% |
| 7 | Potassium Azeloyl Diglycinate | 5.0% |
| 8 | Fragrances | 0.3% |
| 9 | D-Panthenol | 1.0% |
| 10 | Preservative | 0.2% |

### Formulation Example 2

Cosmetic cream with chemical peeling effect

| | | |
|---|---|---|
| 1 | Aqua | to 100% |
| 2 | Octyl Palmitate | 15% |
| 3 | Cetearyl Alcohol | 3.5% |
| 4 | Gliceryl Stearate | 3.5% |
| 5 | Potassium Olivoyl Hydrolyzed Wheat Protein | 2.5% |
| 6 | Palmitoyl Glucoside | 1.0% |
| 7 | Sodium Azeloyl Glutamate | 5.0% |
| 8 | D-Panthenol | 0.5% |
| 9 | Tocopheryl Acetate | 1.0% |
| 10 | Fragrances | 0.3% |
| 11 | Preservative | 0.2% |

### Formulation Example 3

Whitening and regenerating cosmetic cream

| | | |
|---|---|---|
| 1 | Aqua | to 100% |
| 2 | Octyl Palmitate | 15% |
| 3 | Cetearyl Alcohol | 3.5% |
| 4 | Gliceryl Stearate | 3.5% |
| 5 | Jojoba Oil | 5.0% |
| 6 | Sweet Almond Oil | 5.0% |
| 7 | Perhydrosqualene | 5.0% |
| 8 | Ceteareth 25 | 1.5% |
| 9 | D-Panthenol | 0.5% |
| 10 | Retinyl Palmitate | 0.3% |
| 11 | Achillea Glycolic Extract | 1.0% |
| 12 | Potassium Azeloyl Glutamyl Glicine | 5.0% |
| 13 | Fragrances | 0.3% |
| 14 | Preservative | 0.2% |

### Formulation Example 4

Whitening and regenerating cosmetic cream

| | | |
|---|---|---|
| 1 | Aqua | to 100% |
| 2 | Sweet Almond Oil | 6% |
| 3 | Cetearyl Alcohol | 4% |
| 4 | Gliceryl Stearate | 4% |
| 5 | Jojoba Oil | 6.0% |
| 6 | Perhydrosqualene | 3.0% |
| 7 | Calendula Glycolic Extract | 1.0% |
| 8 | Potassium Azeloyl Proline Arginine | 5.0% |
| 9 | Fragrances | 0.3% |
| 10 | Preservative | 0.2% |

### Formulation Example 5

Freshening and regenerating cosmetic cream

| | | |
|---|---|---|
| 1 | Aqua | to 100% |
| 2 | Octyl Palmitate | 5% |
| 3 | Cetearyl Alcohol | 2% |
| 4 | Gliceryl Stearate | 3% |
| 5 | Jojoba Oil | 5.0% |
| 6 | Aloe Vera Gel | 0.5% |
| 7 | D-Panthenol | 1.0% |
| 8 | Potassium Azeloyl Proline Hydrolyzed Wheat Protein | 5.0% |
| 9 | Ascorbyl Palmitate | 1.0% |
| 10 | Fragrances | 0.3% |
| 11 | Preservative | 0.2% |

### Formulation Example 6

Hydrating sebonormalizing cosmetic cream

| | | |
|---|---|---|
| 1 | Aqua | to 100% |
| 2 | Caprylic Capric Tryglyceride | 5% |
| 3 | Cetearyl Alcohol | 4% |
| 4 | Polygliceryl Isostearate | 2% |
| 5 | Sweet Almond Oil | 8.0% |
| 6 | Glycerin | 1.0% |
| 7 | Tocopheryl Acetate | 1.0% |
| 8 | Malva Glycolic Extract | 1.0% |
| 9 | Azeloyl Bis Hydrolyzed Oat Protein | 5.0% |
| 10 | Fragrances | 0.3% |
| 11 | Preservative | 0.2% |

### Formulation Example 7

Whitening and emollient cosmetic cream

| | | |
|---|---|---|
| 1 | Aqua | to 100% |
| 2 | Jojoba Oil | 5% |
| 3 | Octyl Palmitate | 5% |
| 4 | Cetearyl Isononanoate | 6.0% |
| 5 | Sorbitan Isostearate | 3.0% |
| 6 | Glycerin | 1.0% |
| 7 | Potassium Lauroyl Oat Amino Acids | 1.0% |
| 8 | Potassium Azeloyl Mono Glicine | 1.0% |
| 9 | Retinyl Palmitate | 0.2% |
| 10 | Ascorbyl Palmitate | 0.5% |
| 11 | D-Panthenol | 1.0% |
| 12 | Fragrances | 0.3% |
| 13 | Preservative | 0.2% |

### Formulation Example 8

Whitening and nutrient cosmetic cream

| | | |
|---|---|---|
| 1 | Aqua | to 100% |
| 2 | Caprylic Capric Tryglyceride | 5% |
| 3 | Isopropyl Palmitate | 5% |
| 4 | Cetearyl Isononanoate | 6.0% |
| 5 | Glyceryl Stearate | 3.0% |
| 6 | Retinyl Palmitate | 0.2% |
| 7 | Potassium Lauroyl Hydrolyzed Oat Protein | 1.0% |
| 8 | Azeloyl Bis Hydrolyzed Wheat Amino Acids | 4.0% |
| 9 | Camomilla Glycolic Extract | 1.0% |
| 10 | Fragrances | 0.3% |
| 11 | Preservative | 0.2% |

### Formulation Example 9

Whitening functional cosmetic cream

| | | |
|---|---|---|
| 1 | Aqua | to 100% |
| 2 | Sweet Almond Protein | 5% |
| 3 | Perhydrosqualene | 5% |
| 4 | Cetearyl Alcohol | 6.0% |
| 5 | Glyceryl Stearate | 3.0% |
| 6 | Tocopheryl Acetate | 0.2% |
| 7 | Decyl Oleate | 2.0% |
| 8 | Azeloyl Hydrolyzed Oat Amino Acids | 4.0% |
| 9 | Ginseng Glycolic Extract | 1.0% |
| 10 | Fragrances | 0.3% |
| 11 | Preservative | 0.2% |

1. Compounds of formula (I): 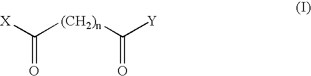
in which:
n is an integer from 2 to 16;
X and Y, which can be the same or different, are OM or a residue of formula MOOCHRNH— wherein R is the residue of a natural alpha-amino acid and M is hydrogen or a non toxic cation, or X and/or Y are a —NH—P residue, wherein P is a polypeptide deriving from the hydrolysis of vegetable proteins, with the proviso that at least one of X and Y is different from OH and that X and Y are not both —NHCH₂—COOK groups when n is 7.

2. Compounds as claimed in claim 1 wherein R is hydrogen or the residue of the amino acids arginine, optionally salified glutamic acid, hydroxyproline, proline.

3. Compounds as claimed in claim 1 in which the vegetable protein hydrolysates P comprise protein hydrolysates of soy, oat, wheat and sweet almond.

4. Compounds as claimed in claim 1 which comprise compounds of formula (I) in which:
both X and Y are a residue of formula MOOCHRNH—, as defined above;
both X and Y are a residue of formula —NH—P, as defined above;
one of X and Y is a residue of formula —NH—P and the other is a residue of formula MOOCHRNH—, as defined above;
one of X or Y is a residue of formula MOOCHRNH— and the other is OH or OM;
one of X or Y is a residue of formula —NH—P and the other is OM;
n is 7.

5. Compositions comprising a compound of claim 1 as the active ingredient

6. Compositions as claimed in claim 5 for the cosmetic, pharmaceutical and dermatological uses.

7. Compositions as claimed in claim 6 in the form of cream, gel, lotion or foam.

8. Compounds as claimed in claim 2 in which the vegetable protein hydrolysates P comprise protein hydrolysates of soy, oat, wheat and sweet almond.

9. Compounds as claimed in claim 2 which comprise compounds of formula (I) in which:
both X and Y are a residue of formula MOOCHRNH—, as defined above;
both X and Y are a residue of formula —NH—P, as defined above;
one of X and Y is a residue of formula —NH—P and the other is a residue of formula MOOCHRNH—, as defined above;
one of X or Y is a residue of formula MOOCHRNH— and the other is OH or OM;
one of X or Y is a residue of formula —NH—P and the other is OM;
n is 7.

10. Compounds as claimed in claim 3 which comprise compounds of formula (I) in which:
both X and Y are a residue of formula MOOCHRNH—, as defined above;
both X and Y are a residue of formula —NH—P, as defined above;
one of X and Y is a residue of formula —NH—P and the other is a residue of formula MOOCHRNH—, as defined above;
one of X or Y is a residue of formula MOOCHRNH— and the other is OH or OM;
one of X or Y is a residue of formula —NH—P and the other is OM;
n is 7.

11. Compositions comprising a compound of claim 2 as the active ingredient.

12. Compositions comprising a compound of claim 3 as the active ingredient.

13. Compositions comprising a compound of claim 4 as the active ingredient.
